(19) United States
(12) Patent Application Publication
(10) Pub. No.: US 2004/0091883 A1
(43) Pub. Date: May 13, 2004

(54) Method for analysing and displaying ORF as well as UTR in cDNA sequences and its application to protein synthesis

(75) Inventors: Kouichi Kimura, Koganei (JP); Keiichi Nagai, Higashiyamato (JP); Tetsuo Nishikawa, Tokyo (JP)

Correspondence Address: Stanley P. Fisher, Reed Smith LLP, Suite 1400, 3110 Fairview Park Drive, Falls Church, VA, 22042-4503, US

(73) Assignee: Hitachi, Ltd.

(21) Appl. No.: 10/361,927

(22) Filed: Feb. 11, 2003

(30) Foreign Application Priority Data
Nov. 12, 2002 (JP) 2002-328516

(51) Int. Cl. C12Q 1/68; G06F 19/00; G01N 33/48; G01N 33/50

(52) U.S. Cl. 435/6; 702/20

(57) ABSTRACT

An area is estimated and displayed of a defective translated region of protein included in either one of a cDNA sequence originating from an immature mRNA, and a truncated cDNA and the like. By means of learning results using known mRNA sequence data, likelihood that there is either one of a translated region and untranslated region at each position in a nucleotide sequence is tested locally, and also a similarity analysis with the known proteins and genome sequences is executed upon whereby the results of the analysis thereabove is exhibited along the nucleotide sequence coordinate for simultaneous comparison.

### BACKGROUND OF THE INVENTION

1. Field of the Invention

The present invention relates to a method for analyzing information relating to a gene sequence, and a method in which a region to code protein from cDNA nucleotide sequence data is estimated, and to displaying a coding potential representing a code region in each base position. Specifically, the present invention relates to an effective analysis method for a cDNA sequence not containing a complete translated region of protein, for example, a truncated cDNA sequence, and a cDNA sequence originating from an immature mRNA.

2. Description of the Related Arts

Genetic information of organisms is stored within genome as a DNA sequence and when required a portion of that region is transcripted and spliced into mRNA. Furthermore the portion of sequence thereof is translated into protein which is an amino acid sequence, and a plurality of these protein functions cooperatively, and are expressed in vivo. In following this, in order to examine gene information expressed in vivo the expressed mRNA is extracted then reverse transcribed into a more stable cDNA sequence, and amplified by PCR (Polymerase Chain Reaction), and thus the nucleotide sequence is defined by the use of a sequencer. Directly defining an amino acid sequence of protein is comparative to defining a nucleotide sequence of a genome or cDNA sequence, and since this is technically quite difficult, as well as being expensive, it is standard to obtain an amino acid sequence of protein by way of translation.

In order to translate a nucleotide sequence formed by a group of 4 types of bases, A, G, C and T into an amino acid sequence formed by a group of 20 types of amino acids the nucleotide sequence is segmented into groups of 3 letters from one specific position (translation initiation position) within the nucleotide sequence to another specific position (translation termination position), and therefore a 3 letter nucleotide made to correspond to a 1 letter amino acid can be obtained. A table in which 64 combinations (4×4×4) of 3 letter nucleotides are made to correspond to 1 letter amino acids is called a codon table and combinations thereof are common to most organisms. In a translation initiation position there is ATG (initiation codon) and in a translation termination position there is a termination codon of either one of TAA, TGA and TAG. Though not only does ATG correspond to methionine an amino acid, only a specific ATG is used as an initiation codon, but ATG other than the ATG therebefore, corresponds to methionine when appearing midway through a translation. Whereas TAA, TGA and TAG do not correspond to amino acid and always function as termination codons.

Generally, there are 3 types of methods for segmenting nucleotide sequences into groups of 3 letters. The segmenting types thereof are called reading frames. A reading frame is determined by an initiation codon position. When a nucleotide sequence is given, until either one of TAA, TGA and TAG which are segmented into 3 letters each from a given ATG that appears therein first appears a subsequence containing a number of nucleotides which is a multiple of 3 is called an ORF (Open Reading Frame). Although there is numerous ORF within a cDNA nucleotide sequence, normally only one ORF of the ORF within vivo are actually translated.

It is generally said that in order to obtain a translated region of protein of a cDNA sequence of prokaryote, including human, that the longest ORF should be obtained. Furthermore, precision can be enhanced by using a test following Kozak rule or a test of a generalized version thereof which uses a weight matrix reflecting expression frequency of the nucleotide sequences initiation codon area. These methods go well in most cases if the CDNA sequence is derived from a complete mRNA, in other words, in the case that a single continues translated region of protein is contained therein.

However, many time an appropriate ORF is not found in the cDNA sequence obtained by actual sequencing. The following can be given as reasons thereof.

1. The cDNA was derived from immature mRNA which had not completed splicing.

2. 5′-end, or 3′-end or both ends were truncated due to fragmentation during PCR amplification.

3. Frame shift occurred due to the nucleotide being skipped or read twice when the sequencer was reading.

4. A nucleotide misread as a different nucleotide resulted in the initiation codon or the termination codon to be lost or to redundantly appear when the sequencer was reading.

5. Chimera generated between different mRNA was mistakenly analyzed.

6. A fragment of genome with no relation to mRNA was mistakenly analyzed.

In order to analyze these events the following methods are generally used.

a. By statistical analysis of the sequence of bases (for a probability that a portion thereof is coded as protein).

b. By similarities of already known protein sequences (of same and different type organisms).

c. By comparison of gene sequences of a same type of organisms.

The type of event happening is hinted at by each of the analysis results but it is generally difficult to say that each of these alone provide definitive evidence. A comprehensive determination is made from these results in light of other biological knowledge. Here, when considering probabilities of the various events it is understood that it is useful to have an easily understood format which shows the analyzed results comparatively of each base position within a cDNA sequence.

In light of the aforementioned problems the objective of the present invention is to provide a method that removes errors from within the actual sequence data, which includes a variety of errors, and that extracts translated regions of protein with high precision.

### SUMMARY OF THE INVENTION

In the present invention where the aforementioned should be achieved the likelihood there is either one of a translated region of protein and a untranslated region of protein in each position of the nucleotide sequence is tested for such a cDNA sequence that does not include a complete translated region of protein, thus the likelihood is to be displayed along with the nucleotide sequence coordinate.

More specifically, the display method according to the present invention displays a nucleotide sequence having an untranslated region and a translated region wherein, a first graph displays a sequence coordinate on an abscissa axis and likelihood of a potential untranslated region on an ordinate axis, and a second graph displays a sequence coordinate on an abscissa axis and likelihood of a potential translated region on an ordinate axis, and wherein the first graph and the second graph are displayed along the sequence coordinate by either one means of superimposition and juxtaposition. The display method according to the present invention is characterized by the above.

The first graph has the sequence coordinate including a 5′-end and a 3′-end. The second graph preferably displays the likelihood of the potential translated region for a first reading frame, a second reading frame one base along from the first reading frame and a third reading frame two bases along from the first reading frame.

Also, the graph is preferably displayed so that in the case that the likelihood is positive the likelihood level is displayed as positive, and in the case that the likelihood is negative the likelihood is displayed as negative, and in the case that the likelihood can not be determined to be either positive and negative the likelihood is displayed in the 0 area.

The graph may have a portion sandwiched between a waveform and the abscissa axis filled in. A method for displaying an intron region of the nucleotide sequence in juxtaposition along the sequence coordinate is also useful.

Similarities relating to protein sequences of identical and different organisms can be displayed in juxtaposition along the sequence coordinate. Furthermore, a point of mismatching nucleotide, a nucleotide insertion and a nucleotide deletion between the nucleotide sequence and the genome sequence of a same organism type can be displayed in juxtaposition along the sequence coordinate.

The likelihood for a nucleotide sequence having untranslated and translated regions can be obtained by the equations (1), (2), (3) and (5) to be hereinafter described.

A protein synthesis method according to the present invention comprising the steps of: selecting one cDNA from a cDNA library that includes a plurality of cDNA; defining a nucleotide sequence of the aforementioned selected cDNA; testing the likelihood of a potential translated region and the likelihood of a potential untranslated region of protein for the obtained nucleotide sequence data; displaying the tested values of the likelihood of a potential translated region of protein and the likelihood of a potential untranslated region by means of a method of one of the claims according to any one of claims 1-8; determining whether a complete translated region of protein is included in the cDNA selected by means of the aforementioned results; and synthesizing a protein transduced into an expression vector in the case that a complete translated region of protein is included in the selected cDNA.

According to the present invention, by comparing test values of local likelihood, similarities analysis results with known proteins and similarities analysis results with genome sequences a determination with high reliability can be made.

### BRIEF DESCRIPTON OF THE DRAWINGS

FIG. 1 is a schematic diagram illustrating the entire procedure according to an embodiment of the present invention.

FIG. 2 is a schematic diagram illustrating a process where parameters are learned for local likelihood of each separate region.

FIG. 3 is a diagram explaining a 5′UTR, a translated region, a 3′UTR, an initiation codon and a termination codon.

FIG. 4 is a diagram showing an example for the purpose of explaining a reading frame and a site.

FIG. 5 is a diagram showing an example of a k-tuple frequency table.

FIG. 6 is an explanatory diagram showing an example display of analysis results according the embodiment of the present invention.

FIG. 7 is a diagram showing an example for the purpose of explaining the usefulness of a graph displaying local likelihood.

FIG. 8 is a diagram showing an example for the purpose of explaining the usefulness of a graph displaying similarities between protein sequences.

FIG. 9 is diagram showing an example for the purpose of explaining the usefulness of a graph 680 displaying differences between a CDNA sequence and a genome sequence.

FIG. 10 is a diagram showing steps from obtaining mRNA until generation of protein applied in a test method according to the present invention.

### DETAILED DESCRIPTION OF THE PREFERRED EMBODIMENT

In the present invention, in relation to a given cDNA sequence, a method consisting of the following processing steps shows useful information and by displaying the various analysis results of each base position of the cDNA sequence. Hence a user is able to make presumptions from a translated region of protein and is able to test the probability that a translated region of protein has been lost due to various events.

Step (1) includes the following steps where mRNA sequences are gathered from within the public database this includes completely translated regions of protein that are known, and are divided into two sets, the learning data set and the test data set.

In step (1-1), in relation to the learning data set and the test data set of each mRNA sequence, the sequence thereof is divided into three regions: a 5′UTR (5′ untranslated region, upper untranslated region), a translated region of protein, and a 3′UTR (3′ untranslated region, lower untranslated region).

In step (1-2), an integer of k is at level between 5 and 9, in relation to length k of every nucleotide sequence (k-tuple), the occurrence frequency k-tuple is counted in the learning data set of 5′UTR and 3′UTR of the mRNA sequence and well as the entire mRNA sequence. Furthermore, when there is an occurrence of k-tuple in the translated region of protein of the learning data set, the number of the position (site) that the base occupies of the codon for the base in the last position of the k-tuple is obtained, and the occurrence frequency of k-tuple for each of the sites 1, 2 and 3 in the translated region of protein is counted.

In step (1-3), in relation to 5′UTR, 3′UTR and each site of the translated region of protein as well as each separate region of the entire mRNA sequence, a conditional probability table (transition probability) which shows where the next base appears under conditions, is calculated from a table showing k-tuple occurrence frequency.

In step (1-4), learning data parameters of local likelihood appearance are obtained of the next appearing base under conditions of (k−1)-tuple in relation to 5′UTR, 3′UTR and each translated region of protein for each site and where the transitional probability relating to 5′UTR, 3′UTR and each translated region of protein for each site is compared to the transitional probability in the entire mRNA sequence.

In step (1-5), totals are obtained of, the local likelihood for appearance of the next base under (k−1)-tuple conditions in each base position within the 5′UTR, the local likelihood for appearance of the next base under (k−1)-tuple conditions in each base position within the 3′UTR, the local likelihood for appearance in the site of the next base under (k−1)-tuple conditions in each base position within the translated region of protein. The sum of these totals is then summed up to calculate the local likelihood of the translated region of protein.

In step (1-6), in relation to the test data set of each mRNA sequence, every ORF is considered and calculated in a similar manner to the preceding paragraph and the local likelihood is obtained as the ORF of the translated region of protein.

In step (1-7) in relation to the test data set of each mRNA sequence, the reliability of the local likelihood values for the appearance of the next base under (k−1)-tuple conditions is obtained in each region by comparing the preceding paragraph and the paragraph preceding that and by calculating the ratio of the mRNA sequence for the local likelihood of translated regions of protein which have a larger value than the local likelihood of the ORF thereabove.

In step (2), with the assumption that each base position of a given cDNA sequence is 5′UTR the local likelihood for the appearance of the next base under (k−1)-tuple conditions is calculated and a low pass filter is applied for the smoothing of the values of the laid out order of base positions. Then these values are displayed in line with the cDNA sequence coordinates.

In step (3), with the assumption that each base position of the given cDNA sequence is 3′UTR the local likelihood for the appearance of the next base under (k−1)-tuple conditions is calculated and a low pass filter is applied for the smoothing of the values of the laid out order of base positions. Then these values are displayed in line with the cDNA sequence coordinates.

In step (4), in relation to each of reading frames 1, 2 and 3, with the assumption that each base position of the given cDNA sequence is the reading frame of the translated region of protein, the local likelihood for the appearance of the next base under (k−1)-tuple conditions is calculated and a low pass filter is applied for the smoothing of the values of the laid out order of nucleotide positions. Then these values are displayed in line with the cDNA sequence coordinates.

Step (5) includes the following steps where similarities in the translated sequences of the given cDNA sequence are searched for in relation to a database which has a collection of known protein sequences of the same and different organisms.

(5-1) is a step to identify what subsequence area of a given cDNA is to be translated into a similar sequence of a subsequence of a known protein sequence for each protein sequence found, and to obtain the identity value (a rate of concordance of the amino acid sequence) and the reading frame of the subsequence thereof.

In step (5-2), segments of subsequences having an identity value over a threshold are extracted and those segments are displayed in line with the sequence coordinates, where segments thereof corresponding to the same protein sequence have the same y coordinates and where the reading frames are definitely indicated with colors and lines.

Step (6) includes the following steps in which similar sequences are searched for which possess a high degree of similarity within a given cDNA sequence in relation to a public database which has a collection gene sequences of a same type.

(6-1) is a step to identify what subsequence area of a given cDNA has high similarities to that of a subsequence of a genome sequence for each genome sequence found, if there are mismatched portions therein, the portions thereof are investigated to ascertain whether each respective portion is a position of replacement, insertion or deletion. Depending on the aforementioned the cDNA sequence and the gene sequence is then investigated to check whether a discrepancy has arisen in the initiation codon or the termination codon or not.

In step (6-2), segments of subsequence of the genome sequence having a high degree of similarity are displayed by lines along the cDNA sequence coordinates, to have the same y coordinates as those segments corresponding to the same genome sequence. Both ends display points which correspond to the borders of exon and intron. The insertion and deletion positions within the segments are indicated by a different type of point as possibly being frame shift positions. The positions where errors have arisen in the initiation codon or the termination codon of the cDNA sequence and the genome sequence are indicated with one more different type of point.

In step (7), the area between 0 (horizontal axis) is filled in on graphs (3), (4) and (5) so as to clearly distinguish which segments are positive and which are negative for the relative log likelihood which has a low pass filter applied thereon.

Detailed description of the preferred embodiments in accordance to the present invention will be given below with reference to the drawings.

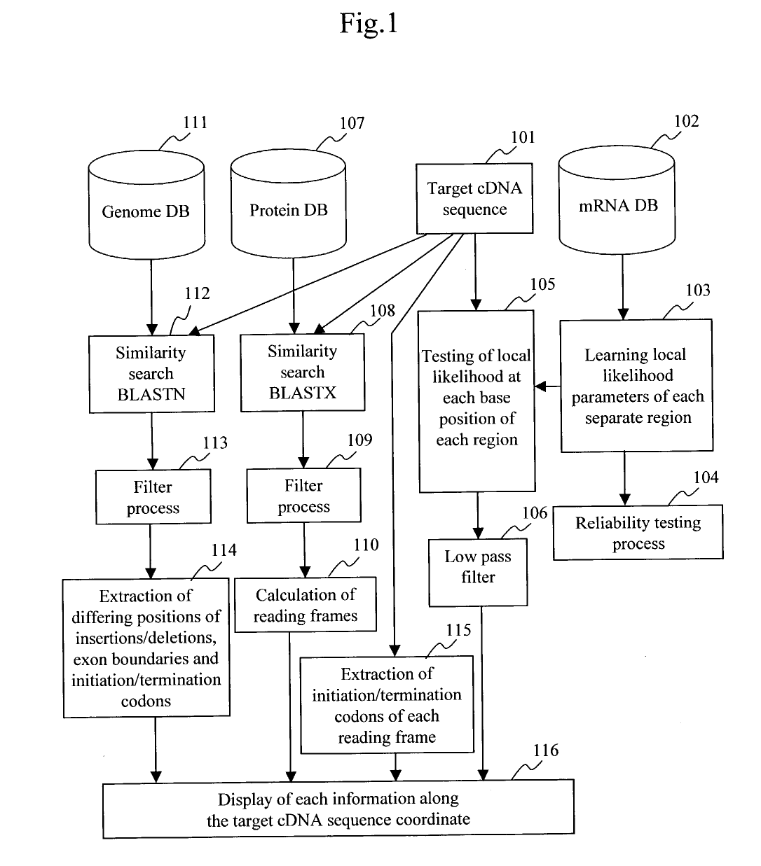

FIG. 1 shows a summary of processes according to an embodiment of the present invention. The reference numeral 101 is target cDNA sequence data to be analyzed. mRNA DB 102 is a public database of known mRNA organism type targeted for analysis. For example, the RefSeq database of the U.S. National Center for Biotechnology Information (NCBI) can be used. Process 103 is a process to learn parameter likelihood for testing whether a line of local nucleotide sequence from the database 102 of known mRNA sequence information correspond to a translated region of protein or an untranslated region of protein. Process 104 is a process to test reliability of resulting learnt parameters from process 103. Process 105 is a process that takes the resulting learnt parameters of local likelihood from process 103 based on each base position of the target cDNA sequence 101 to test whether that base position corresponds to a translated region of protein or an untranslated region of protein. Process 106 is a process that takes the test values obtained of local likelihood from process 105 and a low pass filter is applied over the arranged base positions. As a low pass filter a publicly known Butterworth filter can be applied.

Database 107 is a database of known protein amino acid sequence with same or different types of organisms as the target of analysis. For example, the nr database of NCBI can be used. Process 108 is a process which searches for similarities between the target cDNA sequence 101 and the protein sequence database 107, recognizing even the slightest similarities. This search, while translating protein sequence into amino acid sequence searches out segments which possess similarities. This is made possible by using publicly known technology, for example by using BLASTX (Altschul, Stephen F., Thomas L. Madden, Alejandro A. Schaffer, Jinghui Zhang, Zheng Zhang, Webb Miller, and David J. Lipman (1997), “Gapped BLAST and PSI-BLAST: a new generation of protein database search programs”, Nucleic Acids Res. 25:3389-3402.) of NCBI. Filter process 109 is a process that discards segments found in process 108 which are below a set threshold for the identity value. Process 110 is a process which searches for the translated reading frames of those similar segments that remained after filter process 109.

Genome DB 111 is a database of genome sequences with same or different organism types of the target analysis. For example, the GenBank database of NCBI can be used. Process 112 is a process which searches for similarities between the target cDNA sequence 101 and the genome sequence database 111. This search is a process for seeking out segments having similarities amongst nucleotide sequences. This is possible by using publicly known technology, for example, by using BLASTN of NCBI. Filter process 113 is a process for keeping only segments with extremely high similarities. Process 114 is a process for making comparison amongst genome and cDNA segments with similarities, and then to extract positions of base insertion/deletion positions, exon border positions, initiation and termination codons that differ therein. Process 115 is a process where all initiation codons and termination codons of each reading frame of the 101 cDNA sequence are extracted. Process 116 is a process that displays the obtained analysis results from processes 106, 110, 114 and 115 in line with the target cDNA sequence 101 sequence coordinates, thus allowing simultaneous comparison.

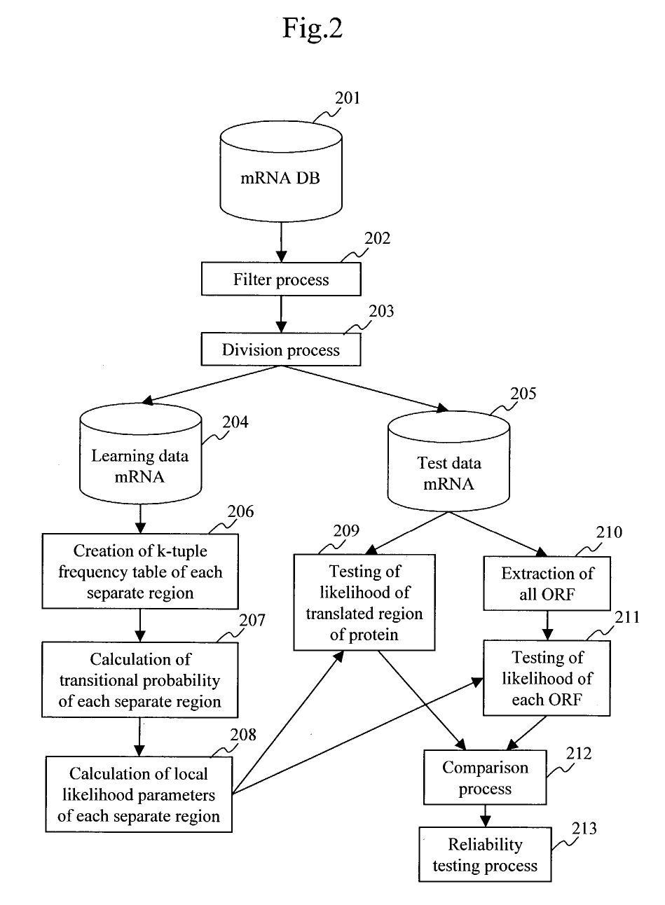

FIG. 2 shows a summary of resulting learnt parameters of local likelihood from process 103 in FIG. 1. mRNA DB 201 is a known mRNA public database which corresponds to mRNA DB 102 of FIG. 1. Filter process 202 is a process which selects out an appropriate mRNA sequence in accordance with learnt parameters. Division process 203 is a process for dividing the selected mRNA sequence into learning data set 204 and test data set 205. For the division of the learning data set 204 and the test data set 205 it is satisfactory, for example, for the entire body to be divided equally. However the division should not be statistically unbalanced, for example, it is necessary to make the division using pseudorandom numbers. Process 206 is a process to create a frequency table that counts the number of occurrences of all k-tuple in each sites translated, untranslated and entire region of protein for the mRNA sequence learning data. Here k is an integer at a level between 5 and 9, where length k of a nucleotide sequence is called k-tuple. Since k-tuple is as much as 4 to the power of k, if the value of k is too small then k-tuple is unable to express the diversity of the nucleotide sequence. Furthermore, in the reverse, if the value of k is too large, nearly all k-tuple frequencies will be 0 thus a frequency table would be unable to be created. Process 207 is a process to calculate a table showing conditional probability (transitional probability) of the next appearance of a base under a (k−1)-tuple condition. Process 208, is a process to obtain local likelihood of the next appearance of a base under a (k−1)-tuple condition in each separate region. This value is a resulting learnt parameter.

Process 209 is a process which tests local likelihood of translated region of protein utilizing the resulting learnt parameter from process 208 for each mRNA sequence of test data mRNA 205. Process 210 is a process for extracting all ORF outside of the translated region of protein for each mRNA sequence of test data mRNA 205. Process 211 is a process for testing local likelihood of the translated region of protein in a similar manner to process 209 for each ORF extracted in process 210. Process 212 is a process where test results of process 209 and process 210 are compared, and where test results of ORF inside and outside the translated region of protein and ORF are compared. Process 213 is a process for testing reliability for learnt parameters obtained in process 208 based on the results of the comparison process from process 212.

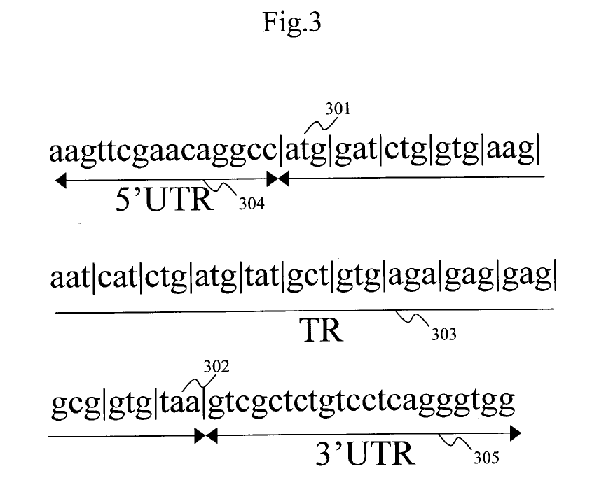

The content of filter process 202 in FIG. 2 will be explained using the mRNA nucleotide sequence shown in FIG. 3 as an example. Firstly, in relation to each mRNA recorded in a database a search is executed to determine whether or not the translated region of one mRNA thereof is listed as being intact. For example, if this was RefSeq database of NCBI, with p and q as positive integers, a CDS item would take the form p..q. p and q here indicate what number position base from the top of the mRNA sequence are the initiation codon and the termination codon. In the example in FIG. 3 the initiation codon is shown by reference numeral 301 and the termination codon shown by reference numeral 302. As shown by reference numeral 303 the region between the initiation codon and the termination codon is referred to by TR (translation region). Furthermore, as shown by reference numeral 304 the portion before the initiation codon is referred to by 5′UTR (5′untranslated region), and the portion following the termination codon is referred to by 3′UTR (3′untranslated region). As shown in the diagram, the nucleotide sequence within the translated region 303 is segmented into groups of 3 bases each which is referred to as a codon, and each of the codon thereof are translated into specific amino acids in accordance to a codon table. In filter process 202 in FIG. 2, only one complete translated region is reportedly included, all the 5′UTR, the translated region and the 3′UTR regions over a threshold, for example including 50 or more bases, are selected and the remaining is discarded. This threshold value is set so that learnt parameters for each region can be utilized efficiently.

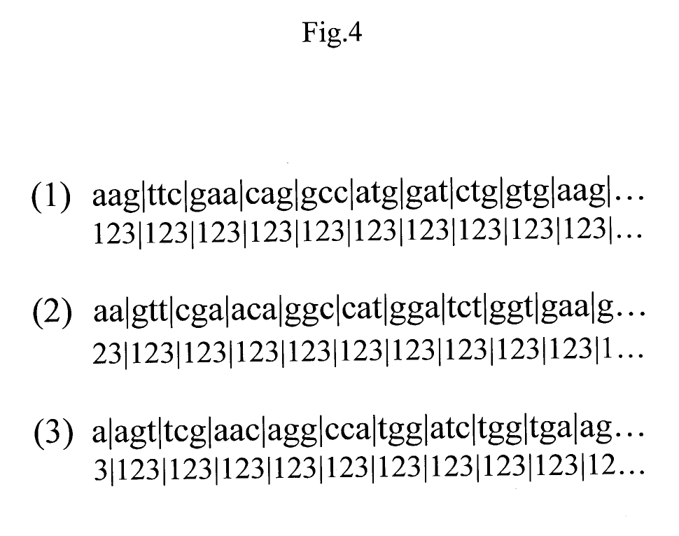

With reference to FIG. 4, the reading frames used when translating a nucleotide sequence into amino acid sequence will be explained, and then a method used to classify base positions into 3 site types when a reading frame has been assumed will be explained. Firstly, since the nucleotide sequence is segmented into codons of 3 bases each to be translated into amino acid, as shown in the diagram there are 3 methods for translating the nucleotide sequence. In the case of (1) in the diagram, when the base position at the head of each codon counted from the top of the nucleotide sequence equals 1 when divided by 3 then that is referred to as reading frame 1. Similarly, in the case of (2) and (3), the methods are referred to as reading frame 2 and reading frame 3 respectively. Next, when a reading frame has been assumed, each base position is either the first base, the second base or the third base within the codon depending on what number position the base thereof is. The base position aforementioned is referred to as site 1, site 2 and site 3. In FIG. 4, the numerals 1, 2 and 3 under each base shows the site number of the base position thereof.

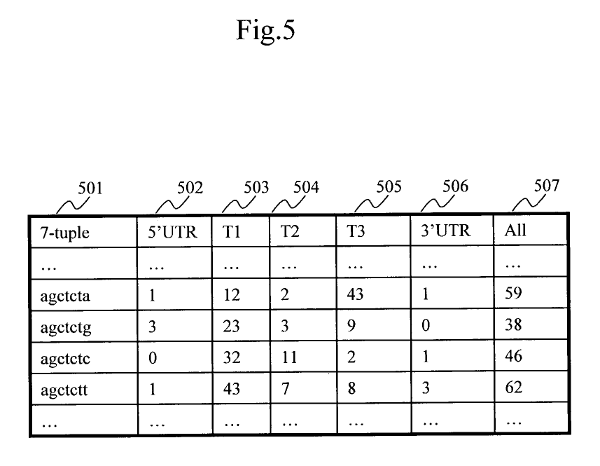

Process 206 is a process for creating a k-tuple frequency table such as that shown in FIG. 5. FIG. 5 shows an example k-tuple frequency table for the translated, untranslated or entire protein region where k=7. Column 501 is a column having an array of every 7-tuple. Column 502 is the number of times of the occurrence of corresponding 7-tuple in 5′UTR. Column 503 is the number of times in which site 1 occurs in the final base position of a translated region under 7-tuple. Similarly, columns 504 and 505 are the number of times in which sites 2 and 3 occurs in the final base position of a translated region under 7-tuple respectively. Column 506 is the number of times of the occurrence of corresponding 7-tuple in 3′UTR. Column 507 is the total number of occurrences within the mRNA sequence regardless of region under 7-tuple.

The transitional probability table of column 507, based on the k-tuple occurrence frequency table for each separate region of process 206, is calculated according to the following equation.

$$\begin{matrix}\begin{matrix}{P}_{R} ( \begin{matrix}{n}_{1} & {n}_{2} & … & {n}_{k - 1} & {n}_{k}\end{matrix} ) = [ {N}_{R} ( \begin{matrix}{n}_{1} & {n}_{2} & … & {n}_{k - 1} & {n}_{k}\end{matrix} ) + 1 / 2 ] / \\ {N}_{R} ( \begin{matrix}{n}_{1} & {n}_{2} & … & {n}_{k - 1} & *\end{matrix} )\end{matrix} & ( 1 ) \\ \begin{matrix}{N}_{R} ( \begin{matrix}{n}_{1} & {n}_{2} & … & {n}_{k - 1} & *\end{matrix} ) = [ {N}_{R} ( \begin{matrix}{n}_{1} & {n}_{2} & … & {n}_{k - 1} & a\end{matrix} ) + 1 / 2 ] + \\ [ {N}_{R} ( \begin{matrix}{n}_{1} & {n}_{2} & … & {n}_{k - 1} & g\end{matrix} ) + 1 / 2 ] + \\ [ {N}_{R} ( \begin{matrix}{n}_{1} & {n}_{2} & … & {n}_{k - 1} & c\end{matrix} ) + 1 / 2 ] + \\ [ {N}_{R} ( \begin{matrix}{n}_{1} & {n}_{2} & … & {n}_{k - 1} & t\end{matrix} ) + 1 / 2 ]\end{matrix} & ( 2 ) \\ ( R = {5}^{′} UTR , T1 , T2 , T3 , {3}^{′} UTR , All ) & \end{matrix}$$

Here, each ni represents either one of a, g, c and t, n1n2 . . . nk represents k-tuple, NR represents a tuple frequency of a region R, PR represents a conditional probability (transition probability) which shows where the next base appears under (k−1)-tuple conditions for a region R. The reason that 1/2 is included midway through the equation is to deal with a situation when the frequency is 0 in following Jeffreys-Perks Law.

The likelihood parameters of each separate region in process 208 is calculated in accordance with the following equation.

$$\begin{matrix}{L}_{R} ( \begin{matrix}{n}_{1} & {n}_{2} & … & {n}_{k - 1} & {n}_{k}\end{matrix} ) = log {P}_{R} ( \begin{matrix}{n}_{1} & {n}_{2} & … & {n}_{k - 1} & {n}_{k}\end{matrix} ) - \\ log {P}_{All} ( \begin{matrix}{n}_{1} & {n}_{2} & … & {n}_{k - 1} & {n}_{k}\end{matrix} ) \\ ( R = {5}^{′} UTR , T1 , T2 , T3 , {3}^{′} UTR )\end{matrix} \qquad ( 3 )$$

The likelihood test value of the translated region of protein for the test data mRNA sequence is calculated according to the following equation.

$$\begin{matrix}M ( p , q ) = sum\_ [ i = k , … , p - 1 ] {L}_{{5}^{′} UTR} ( n ( i - k + 1 , i ) ) + \\ sum\_ [ i = p + k - 1 , … , q ] {L}_{Ts ( i )} ( n ( i - k + 1 , i ) ) + \\ sum\_ [ i = q + k , … , L ] {L}_{{3}^{′} UTR} ( n ( i - k + 1 , i ) )\end{matrix} \qquad ( 4 )$$

Here, n(i−k+1) is a subsequence of length k which is a position i−k+1 from the top of the test data mRNA sequence until a position i, and L is an entire nucleotide sequence length. p and q represents what number position a base is in from the top of the mRNA sequence, that is the initiation codon sites 1 and termination codon sites 2 respectively, sum ₁₃[i=1, . . . , J] represents the total of i=1, 1+1, . . . , J. Furthermore, s(i) represents a base site that in a position i from the top of the mRNA sequence within the translated region.

In the extraction process of all the ORF in process 210 for the test data mRNA sequence all of the occurrence positions of ATG are obtained and then following which the first to appear out of TAA, TAG, and TGA or, the first to appear out of TAA, TAG and TGA before the rear end (3′UTR) of the mRNA sequence, or from the front end (5′UTR) of the mRNA sequence, or the first to appear before the rear end (3′UTR) through all of these sections are obtained.

The calculation of local likelihood of ORF in process 211 is similar to that of 209 where p and q are the first and last base of every ORF and the number of the base position from the top of the cDNA sequence is obtained by formula (4).

The calculation process 212 compares the magnitudes between the test value of local likelihood of the translated region of protein obtained in process 210 and the test value of local likelihood for ORF other than those obtained in process 211. If the local likelihood parameters learnt in process 208 are appropriate, the test value of local likelihood of the translated region of protein obtained in process 210 should be bigger.

In process 213, the ratio of what portion the aforementioned test value of local likelihood of the translated region of protein obtained in process 210 represents within the total is calculated. This value represents the reliability of local likelihood parameters learnt in 208, and the learnt result is considered to be generally reliable if that value is at a level around 0.8 to 0.9 or greater. If the value is not at this level then a size of k of the tuple needs to be modified, or, filter process 202 needs to be reviewed and the threshold value of each regions length of the mRNA utilized for learning needs to be reviewed, or, the information within the mRNA database needs to be reviewed and have inappropriate mRNA (for example, a function which has not been experimentally identified) removed, and it is then necessary to relearn the parameters. Test value C_{R}(i) of the local likelihood for each region R in a position at base position number i from the top of the target cDNA sequence is calculated by the following equation.

C_{R}(i)=L_{R}(n(i−k+1,i) )(R=5′UTR, T1, T2, T3, 3′UTR, i=k, k+1, . . . ,L) (5)

Here, n(i−k+1) is a subsequence of length k which is from a position i−k+1 from the top of the targeted mRNA sequence analysis until a position i, and where L is an entire nucleotide length of mRNA.

Low pass filter process 106 is processed for each region R of 5′UTR, T1, T2, T3 and 3′UTR in which a sequence of numbers can be formed by arranging local likelihood obtained in 105 in order of base position i in following the equation C_{R}(k),C_{R}(k+1), . . . , C_{R}(L) so as to provide an easily viewable graph display where changes can be smoothed out in line with the base position i for the sequence of numbers arranged thereabove, for example, by applying a common-technology-based low pass filter technology such as a Butterworth filter.

In filter process 109, in relation to a cDNA sequence segment and a protein sequence having similarities found in the similarity search of process 108, a resulting translation of the cDNA sequence segment into an amino acid sequence and a protein sequence segment are compared, and the ratio of matching amino acid is calculated as a rate of concordance. Following which, segments having similarities with a rate of concordance above a threshold level approximately 0.4 to 1 are kept, and all other segments are discarded.

In process 110 reading frames of segments of cDNA sequence having similarities within known protein are obtained. Here when the resulting translation of the cDNA sequence segment into the amino acid sequence and the protein sequence segment are compared, the cDNA sequence is shown by one of (1), (2) and (3) of the reading frame in FIG. 4 how codons are segmented.

In filter process 113, only those segments having extremely high similarities are kept and all others are discarded. Here the rate of concordance of base with the similar segments of the cDNA sequence and genome sequence called for is in example 95% and above.

In process 114, by the adjustment of the boundary position of segments of cDNA sequence having similarities in genome sequences of a number of base boundaries of segments having similarities on the genome side corresponding to exon are adjusted and the exon and intron boundaries are made to comply with the so-called GT-AG rule. In following this, the exon boundary position on a cDNA sequence is determined. Furthermore, the corresponding relationship between segments of cDNA sequences having similarities and base segments of genome sequences is investigated, then insertion and deletion positions of bases, mismatching positions of bases and particularly positions in which differences have occurred in initiation codons and termination codons are extracted.

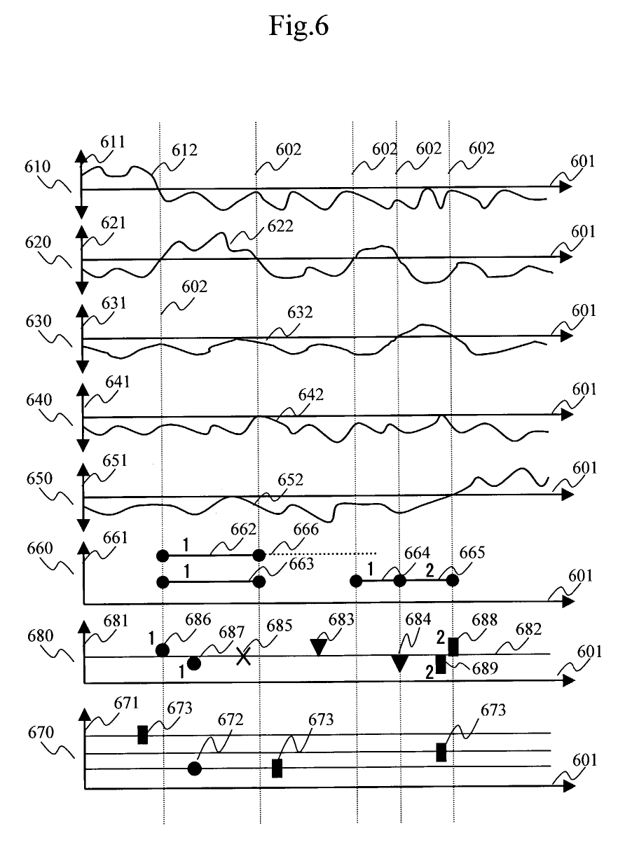

Process 116 is a process that displays the obtained analysis results from processes 106, 110, 114 and 115 in line with the target cDNA sequence coordinates, thus allowing simultaneous comparison, for example, that as displayed in FIG. 6. Graph 610 is a graph in which a low pass filter has been applied to smoothly display the local likelihood which is 5′UTR in that area of each base position of a target cDNA sequence. Similarly, graphs 620, 630 and 640 are each graphs in which a low pass filter has been applied to smoothly display the local likelihood which is the respective translated regions of reading frames 1, 2 and 3 in those areas of each base position of a target CDNA sequence. Graph 650 is a graph in which a low pass filter has been applied to smoothly display the local likelihood which is 3′UTR in that area of each base position of a target cDNA sequence. Graph 660 is a graph that displays segments having similarities in known protein sequences contained within the target cDNA sequence. Graph 670 is a graph that displays positions of initiation codons and termination codons for each reading frame of the target cDNA sequence. Graph 680 is a graph that compares similar target cDNA sequence and the genome sequence and then displays the differences therebetween.

Every graph 610, 620, 630, 640, 650, 660, 670 and 680 share a common cDNA sequence coordinate axis, and as shown in 602 the sequence coordinates are arranged so that events can be compared simultaneously at identical base positions. Coordinate axis 611 is a coordinate axis representing local likelihood of the test value L5′UTR which is 5′UTR and waveform 612 is a resulting plot of L5′UTR that has been smoothed with a low pass filter. Similarly, coordinate axis 621 is a coordinate axis representing the local likelihood of the test value LT1 which is reading frame 1 and waveform 622 is a resulting plot of LT1 that has been smoothed with a low pass filter. Coordinate axis 631 is a coordinate axis representing the local likelihood of the test value LT2 which is reading frame 2 and waveform 632 is a resulting plot of LT2 that has been smoothed with a low pass filter. Coordinate axis 641 is a coordinate axis representing the local likelihood of the test value LT3 which is reading frame 3 and waveform 642 is a resulting plot of LT3 that has been smoothed with a low pass filter. Coordinate axis 651 is a coordinate axis representing local likelihood of the test value L3′UTR which is 3′UTR and waveform 652 is a resulting plot of L3′UTR that has been smoothed with a low pass filter.

Coordinate axis 661 is a coordinate axis to clarify the known protein sequences having similarities in the targeted cDNA sequence analysis. Segment 662 represents one segment having similarities in relation to known protein sequences. Segments 663, 664 and 665 represent all other segments having similarities in relation to known protein sequences other than the foregoing. The numeral attached to each of the segments 662, 663, 664 and 665 indicates the reading frame where the segments have been translated into the protein sequence. Also, 666 represents the length of the sequence remaining (residue) that does not correspond to the cDNA going down from the protein end when alignment is made between segment 662 of the cDNA sequence and known protein sequences. Coordinate axis 671 is a coordinate axis to clarify the 3 different reading frames of the cDNA sequence. Mark 672 represents the initiation codon position and mark 673 represents the termination codon position.

Coordinate axis 680 is a coordinate axis that clarifies genome sequences having high similarities in cDNA sequences. The numeral 682 represents one segments detected with the level of similarity thereof. Mark 683 is a recognized insertion position of a base in the cDNA sequence in comparison to the genome sequence. Mark 684 is a recognized deletion position of a nucleotide in the cDNA sequence in comparison to the genome sequence. Mark 685 indicates a point of mismatch of a base in the genome sequence and the cDNA sequence. Mark 686 represents an initiation codon resulting from the base mismatch that does not often appear in the cDNA sequence side but does in the genome sequence side, and the indicated numeral indicates the reading frame of that case. Similarly, mark 687 represents an initiation codon that does not often appear in the genome sequence side but does in the cDNA sequence side, and the indicated numeral indicates the reading frame of that case. Also, mark 688 represents a termination codon that does not often appear in the cDNA sequence side but does in the genome sequence side, and the indicated numeral indicates the reading frame of that case. Similarly, mark 689 represents a termination codon that does not often appear in the genome sequence side but does in the cDNA sequence side, and the indicated numeral indicates the reading frame of that case.

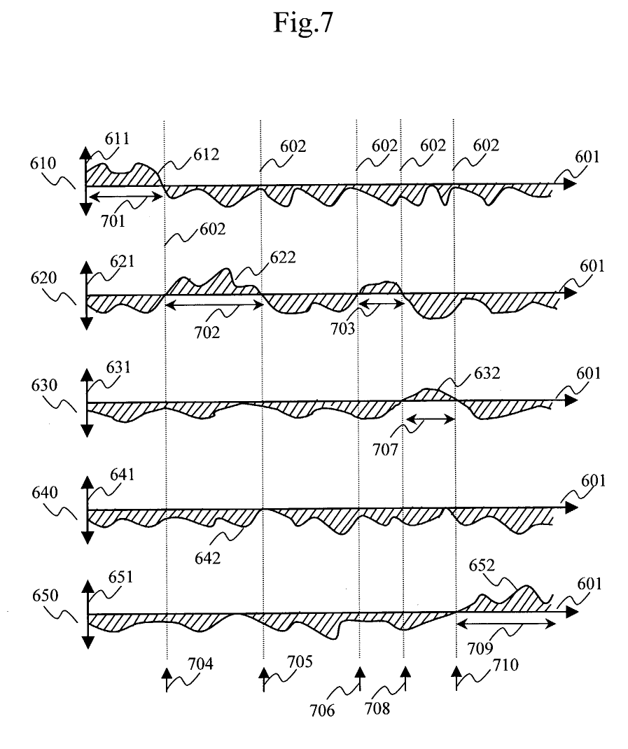

An effectiveness of the present invention will given with reference to the example shown in FIG. 6. FIG. 7 is a portion taken from FIG. 6 having reference numerals added for explanation. Note, the graph, as exemplified by FIG. 7, can have the interior portion of the graph display filled in.

Firstly, in regards to FIG. 7, explanation will be given of the information obtainable by visually comparing the graphs 610 of the local likelihood of 5′UTR and graph 620 of the local likelihood of reading frame 1 thereof. By looking at the resulting plot 612 of L5′UTR which has been smoothed by a low pass filter applied thereon it is understood that a segment indicated by 701 is positive. Similarly, by looking at the resulting plot 622 of LT1 which has been smoothed by a low pass filter applied thereon it is understood that segments indicated by 702 and 703 are positive. By visually comparing the areas indicated by 701 and 702, it can be understood that the base position at 704 is the boundary between both segments. In other words, the local likelihood that is 5′UTR is high in the upper end of 704 (left side of the diagram) and the local likelihood that is the translated region of reading frame 1 is high in the lower end of 704 (right side of the diagram). According to this, it is suggested that an initiation codon is at the position of 704, that 701 is 5′UTR and that 702 is the translated region of reading frame one.

In the segment sandwiched between 702 and 703, each plot 612, 622, 632, 642 and 652 take a negative value, and it is shown that the possibility that this segment is one of 5′UTR, a translated region of reading frame 1, 2 or 3, or 3′UTR is negative. In other words, it is suggested that one possibility other than the aforementioned is that this segment is a segment corresponding to an intron sequence that remained unspliced. Marks 705 and 706 indicate the boundary positions of the intron and exon that remained unspliced.

Next, explanation will be given of the information obtainable by visually comparing the graph 620 of the local likelihood of reading frame 1 and graph 630 of the local likelihood of reading frame 2 thereof. By looking at the resulting plot 632 of LT2 which has been smoothed by a low pass filter applied thereon it is understood that a segment indicated by 707 is positive. By visually comparing the areas indicated by 703 and 707, it can be understood that the base position at 708 is the boundary between both segments. In other words, the local likelihood that is the translated region of reading frame 1 is high in the upper end of 708 (left side of the diagram) and the local likelihood that is the translated region of reading frame 2 is high in the lower end of 708 (right side of the diagram). According to this, it is suggested that frame shift errors occurs due to a deletion at position 708 of a base in the cDNA sequence and that 703 is the translated region of reading frame 1 and that 707 is the translated region of reading frame 2.

Next, the graphs of graph 630 of local likelihood of the reading frame 2 and graph 650 of local likelihood of 3′UTR will be visually compared. By looking at the resulting plot 652 of L3′UTR which has been smoothed by a low pass filter applied thereon, it is understood that a segment indicated by 709 is positive. By visually comparing the areas indicated by 707 and 709, it can be understood that the base position at 710 is the boundary between both segments. In other words, the local likelihood that is the translated region of reading frame 2 is high in the upper end of 710 (left side of the diagram) and the local likelihood that is the translated region of reading frame 2 is high in the lower end of 710 (right side of the diagram). According to this, it is suggested that there is a termination codon at the position 710 and that 709 is 3′UTR.

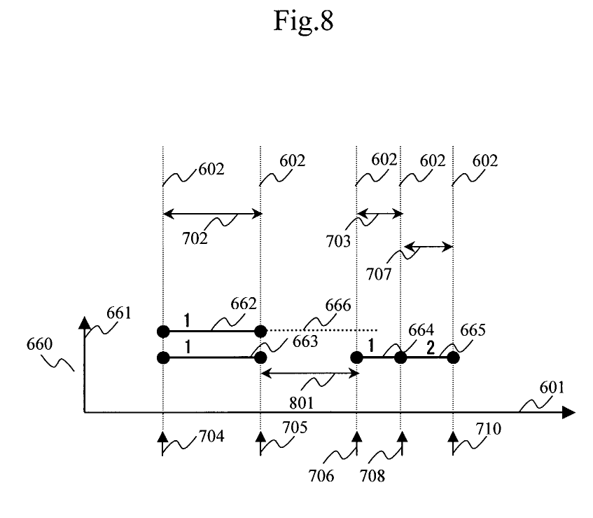

Next, with reference to the example shown in FIG. 6, the usefulness of the graph 660 which displays segments having similarities in known protein sequences will be explained. FIG. 8 is a portion taken from FIG. 6 with a part of the explanation reference numerals used in FIG. 7 added for explanation.

By the local likelihood test of 662 and 663 the segment 702 that is suggested to be the translated region of reading frame 1 verification is shown that the sequence protein coded has similarities.

Similarly, the local likelihood test of 664 and 665 indicates that the segments 703 and 707 that are suggested to be the translated regions of reading frames 1 and 2 respectively are shown that the sequence protein coded in those reading frame has similarities but, at the same time, at position 708 it is shown that there is a change from reading frame 1 to 2 (frame shift) for that same protein sequence. This suggests that at position 708 a base deletion has occurred in the CDNA sequence.

In the alignment between the CDNA sequence and the known protein sequence for 662, because of just the length shown by 666 of sequence remaining that does not correspond to the cDNA in a lower direction from the protein end, it can be seen that this protein does not closely follow the cDNA but is either a protein that originating from a splice variant of this cDNA, or a protein that was derived from a similar gene.

In comparison to this, in the gap between 663 and 664 since no residue arises on the protein sequence end and the protein sequence is matched continuously it is suggested that segment 801 where the residue arose on the cDNA side (not corresponding to the protein sequence) is either an unspliced intron, or that the cDNA sequence is a splice variant of a known protein. The combined with the test results of local likelihood suggest that the latter is not a possibility and that 801 is a remaining unspliced intron.

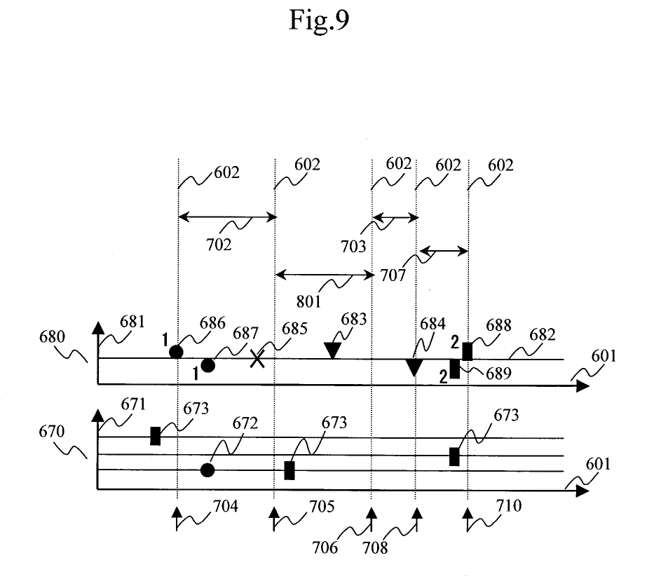

Next, by using the example in FIG. 6 the usefulness of graph 680 is explained comparing the target cDNA sequence and a similar genome sequence and displaying the differences therebetween. FIG. 9 is a portion taken from FIG. 6 with a part of the explanation reference numerals used in FIG. 7 and 8 added for explanation.

The numeral 682 is a wider segment (in this case all segments of the cDNA sequence) than the continuation of the 3 segments 702, 801 and 703 and indicates that the cDNA sequence and the genome sequence have high similarities. In particular, from the similarity analysis of the tested local likelihood and known protein, verification is shown that the segment 801 suggested to be a remaining unspliced intron does correspond to the genome sequence.

The numeral 684 shows a base deletion in the cDNA sequence side that has arisen by position 708 after comparison to the genome sequence. The position 708 is a position which is suggested to be a frame shift occurrence already from the standpoint of the tested local likelihood and from the results of the similarity search with known protein. Here, furthermore it is suggested there is a frame shift occurrence at the position 708 from the standpoint of the genome sequence comparison.

The numeral 686 is the initiation codon of reading frame 1 which is shown to appear in the genome sequence side at the 704 position but not to appear on the cDNA sequence side. At the 704 position it is suggested that the initiation codon of reading frame 1 exists by the test results of local likelihood, but on the graph 670 which displays each of all the initiation codons and the termination codons such an initiation codons existence is not displayed hence there is a discrepancy between the two graphs. However, since the initiation codon of reading frame 1 at the position 704 was found here by comparison with the genome sequence, it is suggested that there was a misread occurrence of the base in the sequencing process of the cDNA sequence at position 704.

The numeral 688 is the termination codon of reading frame 1 which is shown to appear in the genome sequence side at the 710 position but not to appear on the cDNA sequence side. At the 710 position it is suggested that the termination codon of reading frame 2 exists by the test results of local likelihood, but on the graph 670 which displays each of all the termination codons and the termination codons such a termination codons existence is not displayed, hence there is a discrepancy between the two graphs. However, since the termination codon of reading frame 2 at the position 710 was found here by comparison with the genome sequence, it is suggested that there was a misread occurrence of the base in the sequencing process of the cDNA sequence at position 710.

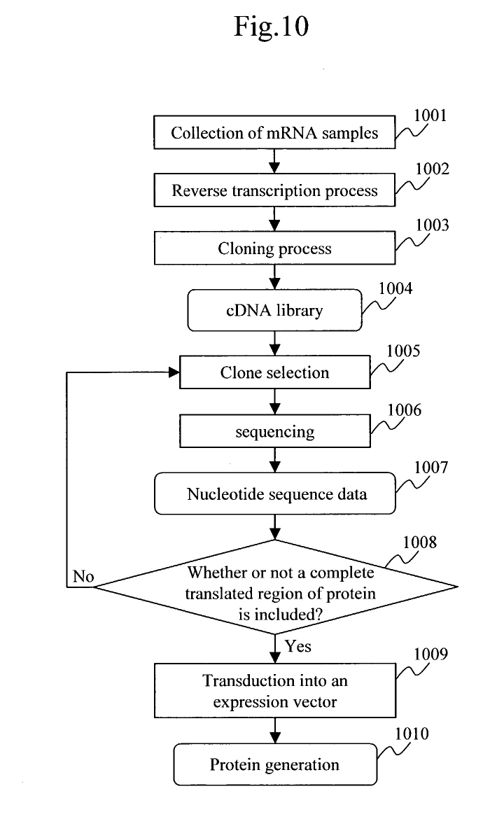

FIG. 10 shows procedures applying the present inventions translated region of protein test method from obtaining mRNA to protein generation. Process 1001 is a process to collect mRNA samples from a living organism cell. Process 1002 is a process to make a reverse transcription of mRNA samples that are easily broken down into a stable cDNA sequence. Process 1003 is a process to amplify the obtained cDNA sequence, and to create cDNA library 1004. Process 1005 is a process to select one clone from the cDNA library which contains numerous clones. Process 1006 is a process to define a nucleotide sequence of the selected clone by use of a sequencer. The translated and untranslated region of protein analyzed for these nucleotide data 1007 in accordance with the procedure in FIG. 1 and analysis results such as those shown in FIG. 6 are obtained. Determination 1008 then determines if the analysis results includes a complete translated region of protein or not, if there is not one included then the process reverts to the clone selection 1005 for reselection. If there is one included, then that complete translated region of protein is transduced into an expression vector as indicated by process 1009 and protein generation 1010 is executed. Every process other than determination 1008 is publicly known technology.

In relation to FIG. 10, by the determination made in 1008, complete protein can be obtained for authentic mRNA. If the determination of 1008 was not made, either a subsequence of authentic protein would not be obtained and the authenticity would be lost, or there would be a complete failure of generation of protein. Therefore, by the present invention, in protein generation the associated risk is decreased, and time and cost can be greatly reduced.

What is claimed is:

1. A display method comprising,
a method for displaying a nucleotide sequence having an untranslated region and a translated region wherein,
a first graph displaying a sequence coordinate on an abscissa axis and likelihood of a potential untranslated region on an ordinate axis, and;
a second graph displaying a sequence coordinate on an abscissa axis and likelihood of a potential translated region on an ordinate axis, and wherein
the first graph and the second graph are displayed along the sequence coordinate by one means of superimposition and juxtaposition.

2. A display method according to claim. 1,
comprising the first graph wherein the sequence coordinate includes a 5′-end and a 3′-end.

3. A display method according to claim. 1,
comprising the second graph wherein likelihood of the potential translated region for a first reading frame, a second reading frame one base along from the first reading frame and a third reading frame two bases along from the first reading frame are displayed.

4. A display method according to claim 1,
comprising the graph display, wherein
in the case that the likelihood is positive, the likelihood is displayed as positive,
in the case that the likelihood is negative, the likelihood is displayed as negative,
and in the case that the likelihood can not be determined to be either positive and negative, the likelihood is displayed in the 0 area.

5. A display method according to claim. 4,
wherein a portion sandwiched between a waveform and the abscissa axis of the graph is filled in.

6. A display method according to claim. 1,
wherein furthermore an intron region of the nucleotide sequence is displayed in juxtaposition along the sequence coordinate.

7. A display method according to claim. 1,
wherein furthermore similarities relating to protein sequences of identical and different organisms are displayed in juxtaposition along the sequence coordinate.

8. A display method according to claim. 1,
wherein furthermore a point of mismatching base, a base insertion and a base deletion are displayed in juxtaposition along the sequence coordinate.

9. A method comprising the step of,
obtaining potential for a nucleotide sequence containing untranslated and translated regions by means of the following equations.
C_{R}(i)=L_{R}(n(i−k+1,i)) (R=5′UTR, T1, T2, T3, 3′UTR, i=k, k+1, . . . ,L)
(here when R=either of T1, T2 and T3, C_{R}(i) is a quantity testing local potential that is a translated region of either one of a first, second and third reading frame for a base position that is i position from the top of the nucleotide sequence, when either one of R=5′UTR and 3′UTR, C_{R}(i) is a quantity testing local potential that is an untranslated region of either one of a 5′-end and a 3′-end for a base position that is i position from the top of the nucleotide sequence, n(i−k+1,i) is a subsequence length k that is formed from a base extending from a i−k+1 of the nucleotide sequence up until an i position and L_{R} is a quantity calculated by means of the following equation.)
$$\begin{matrix}{L}_{R} ( \begin{matrix}{n}_{1} & {n}_{2} & … & {n}_{k - 1} & {n}_{k}\end{matrix} ) = log {P}_{R} ( \begin{matrix}{n}_{1} & {n}_{2} & … & {n}_{k - 1} & {n}_{k}\end{matrix} ) - \\ log {P}_{All} ( \begin{matrix}{n}_{1} & {n}_{2} & … & {n}_{k - 1} & {n}_{k}\end{matrix} ) \\ ( R = {5}^{′} UTR , T1 , T2 , T3 , {3}^{′} UTR )\end{matrix}$$
(Here, P_{R} is a quantity calculated by means of the following equation.)
$${P}_{R} ( {n}_{1} {n}_{2} … {n}_{k - 1} {n}_{k} ) = [ {N}_{R} ( \begin{matrix}{n}_{1} & {n}_{2} & … & {n}_{k - 1} & {n}_{k}\end{matrix} ) + 1 / 2 ] / \\ {N}_{R} ( \begin{matrix}{n}_{1} & {n}_{2} & … & {n}_{k - 1} & *\end{matrix} ) , \\ {N}_{R} ( \begin{matrix}{n}_{1} & {n}_{2} & … & {n}_{k - 1} & *\end{matrix} ) = [ {N}_{R} ( \begin{matrix}{n}_{1} & {n}_{2} & … & {n}_{k - 1} & a\end{matrix} ) + 1 / 2 ] + \\ [ {N}_{R} ( \begin{matrix}{n}_{1} & {n}_{2} & … & {n}_{k - 1} & g\end{matrix} ) + 1 / 2 ] + \\ [ {N}_{R} ( \begin{matrix}{n}_{1} & {n}_{2} & … & {n}_{k - 1} & c\end{matrix} ) + 1 / 2 ] + \\ [ {N}_{R} ( \begin{matrix}{n}_{1} & {n}_{2} & … & {n}_{k - 1} & t\end{matrix} ) + 1 / 2 ] \\ ( R = {5}^{′} UTR , T1 , T2 , T3 , {3}^{′} UTR , All )$$
(Here, when R=All, NR(n1n2 . . . nk) is the number of times in which the nucleotide subsequence n1n2 . . . nk portion of length k for a mRNA sequence data set prepared as test data appears, when R=either one of 5′UTR and 3′UTR, N_{R}(n1n2 . . . nk) is the number of times in which the nucleotide subsequence n1 n2 . . . nk portion of length k for a untranslated region of either one the 5′-end and 3′-end of the mRNA sequence within the data set appears, when R=either one of T1, T2 and T3, N_{R}(n1n2 . . . nk) is the number of times in which the nucleotide subsequence n1n2 . . . nk portion of length k for the translated region the mRNA sequence within the data set appears so that the last base is respectively a first, second and third nucleotide position of a codon.)

10. A protein synthesis method comprising the steps of:
selecting one cDNA from a cDNA library that includes a plurality of cDNA;
defining a nucleotide sequence of the selected cDNA;
testing the likelihood of a potential translated region and the likelihood of a potential untranslated region of protein for the obtained nucleotide sequence data;
displaying the tested values of the likelihood of the potential translated region of protein and the likelihood of the potential untranslated region by means of a method of one of the claims according to any one of claims 1-8;
determining whether a complete translated region of protein is included in the cDNA selected by means of the displaying results; and
for synthesizing a protein transduced into an expression vector in the case that the complete translated region of protein is included in the selected cDNA.


SEQUENCE LISTING (6 sequences)

SEQ ID NO: 1 - DNA, 30 bp, Artificial Sequence
  Description of Artificial SequenceSynthetic DNA
aagttcgaacaggccatggatctggtgaag

SEQ ID NO: 2 - DNA, 30 bp, Artificial Sequence
  Description of Artificial SequenceSynthetic DNA
aatcatctgatgtatgctgtgagagaggag

SEQ ID NO: 3 - DNA, 30 bp, Artificial Sequence
  Description of Artificial SequenceSynthetic DNA
gcggtgtaagtcgctctgtcctcagggtgg

SEQ ID NO: 4 - DNA, 30 bp, Artificial Sequence; identical to SEQ ID NO: 1
  Description of Artificial SequenceSynthetic DNA

SEQ ID NO: 5 - DNA, 30 bp, Artificial Sequence; identical to SEQ ID NO: 1
  Description of Artificial SequenceSynthetic DNA

SEQ ID NO: 6 - DNA, 30 bp, Artificial Sequence; identical to SEQ ID NO: 1
  Description of Artificial SequenceSynthetic DNA